(19) United States
(12) Patent Application Publication
(10) Pub. No.: US 2011/0301628 A1
(43) Pub. Date: Dec. 8, 2011

(54) TECHNIQUES FOR USE WITH A NAIL PENETRATION DEVICE

(76) Inventors: Yossi Gross, Moshav Mazor (IL); Noah Amit, Haifa (IL); Moshe Gelber, Ra'anana (IL); Ruth Alon, Michmoret (IL)

(21) Appl. No.: 13/103,289

(22) Filed: May 9, 2011

Related U.S. Application Data
(63) Continuation-in-part of application No. 12/632,035, filed on Dec. 7, 2009.
(60) Provisional application No. 61/201,044, filed on Dec. 5, 2008.
(60) Provisional application No. 61/418,461, filed on Dec. 1, 2010.

(51) Int. Cl. A61B 17/32

(52) U.S. Cl. 606/169

(57) ABSTRACT

Apparatus and methods are described for cutting a hole in a nail of a subject. The apparatus includes an element having a nail-contacting cutting surface for contacting the subject's nail. A control unit is configured to generate a hole in the subject's nail by moving the nail-contacting surface in a back-and-forth motion, while the nail-contacting surface is in contact with the subject's nail. Other applications are also described.

### CROSS REFERENCES TO RELATED APPLICATIONS

The present application is a continuation-in-part of U.S. patent application Ser. No. 12/632,035 to Alon, filed Dec. 7, 2009, which claims priority from U.S. Provisional Patent Application 61/201,044, entitled “Nail drill,” to Alon, filed Dec. 5, 2008, which is incorporated herein by reference.

The present application claims the benefit of U.S. Provisional Patent Application 61/418,461, entitled “Techniques for use with a nail drill,” to Gross, filed Dec. 1, 2010, which is incorporated herein by reference.

### FIELD OF EMBODIMENTS OF THE INVENTION

Some applications of the present invention generally relate to external medical apparatus. Specifically, some applications of the present invention relate to penetrating a subject's nail.

### BACKGROUND

Onychomycosis is a fungal infection that causes the toenails and/or fingernails to thicken, discolor, and split. The prevalence of onychomycosis in the United States population as a whole is 13%, onychomycosis being more prevalent in the elderly (60%). Onychomycosis can result in permanent nail deformity. The disease has a significant impact on the patient's quality of life (e.g., due to concern regarding the appearance of one's toenails and fingernails, and pain associated with wearing shoes, walking and sports activities).

### SUMMARY OF EMBODIMENTS

For some applications of the present invention, a rotating element (e.g., a saw) is used to generate a hole or a groove in a subject's nail. A nail-contacting surface of the rotating element typically rotates, while the nail-contacting surface is in contact with the subject's nail. The rotation of the surface alternates between being clockwise and counterclockwise. Typically, the clockwise-counterclockwise nature of the motion of the nail-contacting surface prevents damage to the nail bed, which may otherwise be caused by the action of the rotating element. When the nail-contacting surface makes contact with the flexible tissue of the nail bed, the nail-contacting surface simply moves the tissue of the nail bed, but does not cut through the tissue. However, when the nail-contacting surface is in contact with the nail, which is generally rigid, it generates a hole or a groove through the nail.

For some applications, the hole is generated in the nail to relieve pressure associated with a blood-blister underneath the nail. Alternatively, one or more holes are generated to facilitate treatment of onychomycosis. For example, a substance may be administered to the nail bed via the holes for treating the onychomycosis.

For some applications, in order to treat onychomycosis, a plug that is configured to seal a hole that has been generated in a nail of a subject, is placed in the hole. An anti-fungal substance is disposed on a surface of the plug that contacts tissue of the nail bed, thereby treating the nail bed.

There is therefore provided, in accordance with some applications of the present invention, apparatus for cutting a hole in a nail of a subject, including:

an element having a nail-contacting cutting surface for contacting the subject's nail; and

a control unit configured to generate a hole in the subject's nail by moving the nail-contacting surface in a back-and-forth motion, while the nail-contacting surface is in contact with the subject's nail.

For some applications, the control unit is configured to move the nail-contacting surface in the back-and-forth motion, while causing the nail-contacting surface to undergo a net rotation in a direction selected from the group consisting of: clockwise and counterclockwise.

For some applications, the control unit is configured to move the nail-contacting surface in the back-and-forth motion, by moving the nail-contacting surface through back-and-forth motion cycles at a frequency of less than 500 times a minute.

For some applications:

when the nail-contacting surface is initially activated, the control unit is configured to move the nail contacting surface in a single direction for at least two seconds, the single direction being selected from the group consisting of: clockwise and counterclockwise; and
subsequently, the control unit is configured to move the nail-contacting surface in the back-and-forth motion.

For some applications, the apparatus includes a disc-shaped nail-penetration element and the nail-contacting surface includes an outer edge of the disc-shaped nail-penetration element.

There is further provided, in accordance with some applications of the present invention, a method, including:

identifying a thickness of a nail of a subject;

determining a depth to which to penetrate through the nail, in response to the identified thickness;

inputting the determined depth into a control unit of a nail penetration device; and

generating a hole in the nail with the nail penetration device, the control unit being configured to automatically terminate the generation of the hole by the nail-penetration device at the determined depth.

There is additionally provided, in accordance with some applications of the present invention, apparatus including:

a housing;

a plurality of nail penetration elements configured to generate respective holes through a nail of a subject, each of the penetration elements being coupled to the housing at a distance from an adjacent one of the penetration elements that is 1-5 mm;

a sensor unit, coupled to each penetration element and configured to sense a parameter at the distal end of the penetration elements; and

a control unit configured to terminate the generation of a hole by a given one of the penetration elements in response to the parameter that is sensed by the sensor unit indicating that the distal end of the given penetration element is in a vicinity of a nail bed underneath the nail.

For some applications, in response to the parameter that is sensed by any one of the sensor units indicating that the distal end of a corresponding penetration element is in the vicinity of the nail bed, the control unit is configured to terminate the generation of holes by of all of the penetration elements.

There is further provided, in accordance with some applications of the present invention, apparatus for generating a hole in a nail of a subject, including:

a nail penetration device having a nail-contacting surface for contacting the subject's nail, and including a first electrode disposed on the nail-contacting surface;

a skin-contact sensor for detecting contact between the nail-contacting surface and a nail bed of the subject, the sensor including a pair of second electrodes disposable on skin of the subject in a vicinity of the nail; and

a device control unit configured to:
generate a hole in the subject's nail by moving the nail-contacting surface, while the nail-contacting surface is in contact with the subject's nail,
drive a current via the first electrode, the current having a given set of parameters,
detect a voltage between the second electrodes, and
stop the generation of the hole, in response to detecting a change in the voltage between the second electrodes that is indicative of the current that is driven via the first electrode having travelled through the subject's skin to the pair of second electrodes.

There is additionally provided, in accordance with some applications of the present invention, apparatus for generating a hole in a nail of a subject, including:

a nail penetration device having a nail-contacting surface for contacting the subject's nail;

a skin-contact sensor for detecting contact between the nail-contacting surface and a nail bed of the subject, the sensor including an electrode disposable on skin of the subject in a vicinity of the nail; and

a device control unit configured to:
generate a hole in the subject's nail by moving the nail-contacting surface, while the nail-contacting surface is in contact with the subject's nail, and
stop the generation of the hole, in response to detecting a current via the electrode, the current being indicative of stray electrons conducted between skin of the subject's nail bed and the nail-contacting surface.

There is further provided, in accordance with some applications of the present invention, apparatus for generating a hole in a nail of a subject, including:

a nail penetration device having a nail-contacting surface for contacting the subject's nail;

a skin-contact sensor for facilitating detection of contact between the nail-contacting surface and a nail bed of the subject, the sensor including an electrode disposed on the nail-contacting surface; and

a device control unit configured to:
generate a hole in the subject's nail by moving the nail-contacting surface, while the nail-contacting surface is in contact with the subject's nail, and
drive a current via the electrode, the current being configured such that, upon the electrode contacting skin of the subject's nail bed, the current stimulates nerve endings of the nail bed at a level sufficient to cause a sensation by the subject.

For some applications, the device control unit is configured to drive the current via the electrode by driving a current having an amplitude of more than 3 mA.

For some applications, the device control unit is configured to drive the current via the electrode by driving a current having an amplitude of more than 7 mA.

For some applications, the device control unit is configured to drive the current via the electrode by driving a current having an amplitude of more than 8 mA.

For some applications, the device control unit is configured to drive the current via the electrode by driving a current having an amplitude of less than 20 mA.

For some applications, the device control unit is configured to drive the current via the electrode by driving a current having an amplitude of less than 15 mA.

For some applications, the device control unit is configured to drive the current via the electrode by driving a current having an amplitude of less than 12 mA.

For some applications, the device control unit is configured to drive the current via the electrode by driving an AC current having a frequency of more than 50 Hz.

For some applications, the device control unit is configured to drive the current via the electrode by driving an AC current having a frequency of less than 1 kHz.

There is additionally provided, in accordance with some applications of the present invention, apparatus, including:

a plug having a diameter of between 1 mm and 2 mm configured to seal a hole that has been generated in a nail of a subject, by being placed in the hole, the plug having a skin-contact surface that is placed in contact with skin of a nail bed of the nail; and

an anti-fungal substance disposed on the skin-contacting surface, and configured to be administered to the subject by the skin-contact surface being placed in contact with the nail bed.

For some applications, the apparatus further includes a nail penetration device configured to generate the hole in the nail by moving a skin-contacting surface of the nail penetration device in a back-and-forth motion.

For some applications, the apparatus further includes a support element configured to support the plug, the plug being configured to couple the support element to the nail by the plug being placed inside the hole.

For some applications, the support element includes a prosthetic nail.

There is additionally provided, in accordance with some applications of the present invention, apparatus, including:

a nail penetration device configured to generate holes in a nail of a digit of a subject; and

a template configured to be placed on the digit, the template defining a plurality of holes, at least one of the holes being at a distance of less than 10 mm from an adjacent one of the holes, the template being configured to guide the nail penetration device.

For some applications, the template defines a plurality of rows of holes, and the holes are disposed such that holes of one of the rows overlap with holes of at least one other of the rows, across at least 50% of a width of the nail.

For some applications, the template defines between 1 and 12 holes.

For some applications, the holes are within 7 mm of a distal end of the template.

For some applications, upon placement of the template on the digit, the holes are configured to be within 5 mm of a distal end of a nail bed of the digit.

For some applications, the holes are more than 7 mm from a distal end of the template.

For some applications, upon placement of the template on the digit, the holes are configured to be more than 5 mm from a distal end of a nail bed of the digit.

For some applications, the nail penetration device is configured to generate the holes at positions on the nail that are proximal to fungal growth on a nail bed of the digit.

There is further provided, in accordance with some applications of the present invention, a method, including:

creating at least one hole in a nail of a subject; and

coupling a prosthetic nail to the subject's nail, by inserting into the hole a protrusion from a nail-facing surface of the prosthetic nail.

There is additionally provided, in accordance with some applications of the present invention, apparatus including:

a nail penetration element configured to generate a hole in a nail of a subject;

a housing configured to house the nail penetration element, the housing being shaped to accommodate and at least partially surround the nail;

a sensor unit coupled to the nail penetration element and configured to sense a parameter in a vicinity of the distal end of the nail penetration element; and

a control unit configured to terminate the generation of the hole by the nail penetration element in response to the sensed parameter indicating that the distal end of the nail penetration element is in a vicinity of a nail bed underneath the nail.

For some applications, the apparatus further includes a mechanism configured to couple the penetration element to the housing such that:

when the housing is pressed with a pressure that is below a threshold pressure, the penetration element is not activated to penetrate through the nail, and

when the housing is pressed with a pressure that is greater than the threshold pressure, the penetration element is applied to the nail with a constant pressure, irrespective of the extent to which the pressure with which the housing is pressed is greater than the threshold pressure.

For some applications:

the penetration element includes a plurality of nail penetration elements configured to generate respective holes through the nail, each of the penetration elements being coupled to the housing at a distance from an adjacent one of the penetration elements that is 1-5 mm;

the sensor unit includes a plurality of sensor units, each of the sensor units being coupled to one of the penetration elements and configured to sense a parameter at a distal end of the penetration element; and

the control unit is configured to terminate the generation of a hole by a given one of the penetration elements in response to the parameter that is sensed by the sensor unit indicating that the distal end of the given penetration element is in the vicinity of the nail bed.

For some applications, in response to the parameter that is sensed by any one of the sensor units indicating that the distal end of a corresponding penetration element is in the vicinity of the nail bed, the control unit is configured to terminate the generation of holes by of all of the penetration elements.

There is further provided, in accordance with some applications of the present invention, apparatus including:

a penetration element configured to generate a hole through a nail of a subject;

a housing configured to house the penetration element; and

a mechanism configured to couple the penetration element to the housing such that:
when the housing is pressed with a pressure that is below a threshold pressure, the penetration element is not activated to penetrate through the nail, and
when the housing is pressed with a pressure that is greater than the threshold pressure, the penetration element is applied to the nail with a constant pressure, irrespective of the extent to which the pressure with which the housing is pressed is greater than the threshold pressure.

The present invention will be more fully understood from the following detailed description of embodiments thereof, taken together with the drawings, in which:

### BRIEF DESCRIPTION OF THE DRAWINGS

FIG. 1 is a schematic illustration of a nail penetration device for facilitating penetration of a subject's nail, in accordance with some applications of the present invention;

FIG. 2 is a schematic illustration of a substance dispenser for administering a substance into a nail that has been treated by the nail penetration device of FIG. 1, in accordance with some applications of the present invention;

FIGS. 3A-C are schematic illustrations of a template for use with a nail penetration device, in accordance with some applications of the present invention;

FIG. 4A is a schematic illustration of the nail penetration device penetrating the subject's nail at a position that is proximal to fungal growth that is on the nail;

FIG. 4B is a schematic illustration of a placement element for use with a nail penetration device, in accordance with some applications of the present invention;

FIGS. 5A-C are schematic illustrations of respective views of a plug unit for placing over a hole in a nail, in accordance with some applications of the present invention;

FIG. 6 is a schematic illustration of a rotating element for facilitating penetration of a nail, in accordance with some applications of the present invention;

FIG. 7 is a schematic illustration of a housing for use with a nail penetration device, in accordance with some applications of the present invention; and

FIG. 8 is a schematic illustration of a housing that contains a plurality of penetration elements for facilitating the formation of a plurality of holes in a subject's nail, in accordance with some applications of the present invention.

### DETAILED DESCRIPTION OF EMBODIMENTS

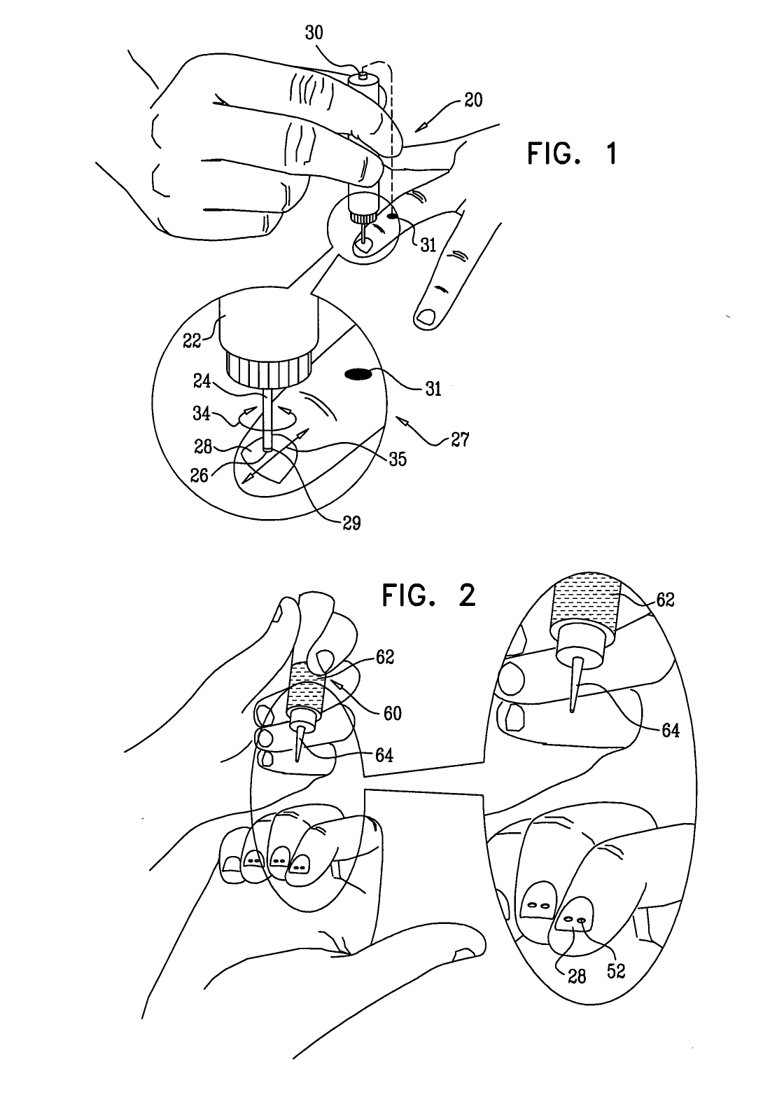

Reference is now made to FIG. 1 which is a schematic illustration of a nail penetration device 20 (e.g., a nail drill), which is generally as described in US Patent Application Publication 2010/0145373 to Alon (except for differences described herein), which is incorporated herein by reference, in accordance with some applications of the present invention. The nail penetration device includes a handle portion 22 and a penetration element 24 (e.g., a drill bit). During operation of the nail penetration device, a user holds the handle portion and pushes a nail-contacting surface 26 of the penetration element against a nail 28. For some applications, in order to activate the nail penetration device, the user presses a power button 30. Alternatively, the nail penetration device functions automatically in response to the nail exerting pressure on the nail-contacting surface. A control unit drives nail-contacting surface 26 to move in a back-and-forth motion, for example, in a rotating back-and-forth motion as indicated by arrow 34, or in a translating back-and-forth motion as indicated by arrow 35. Typically, moving surface 26 in a back-and-forth motion ensures that upon penetrating the nail and making contact with the nail bed (i.e., the skin underneath the nail), surface 26 simply moves the skin of the nail bed, but does not penetrate through the skin or cause the skin any significant trauma.

Typically, handle portion 22 of nail penetration device 20 is configured for multiple uses, and penetration element 24 is configured for single-use and is reversibly couplable to the handle portion. For some applications, the penetration element is at least partially covered with an opaque cap (not shown) (a) so as to obscure the view that the subject has of the nail being penetrated by the penetration element, and/or (b) so as to shield the surroundings of the nail penetration device from nail debris. Typically, for such applications, the opaque cap is configured for single use and is reversibly couplable to the handle portion.

For some applications, the opaque cap does not cover the nail penetration element around the full circumference of the nail penetration element. For example, the opaque cap may (a) cover a first portion of the penetration element so as to obscure the view that the subject has of the nail being penetrated by the penetration element, and (b) not cover a second portion of the penetration element so as not to obscure the view that an operator of the nail penetration device has of the nail being penetrated by the penetration element.

For some applications, nail contacting surface 26 of penetration element 24 is rounded. Typically, the penetration element has a diameter of more than 1 mm and/or less than 3 mm, e.g., about 2 mm. For some applications, the nail penetration element is made of stainless steel.

For some applications, the control unit initially drives nail-contacting surface 26 in a single direction (e.g., clockwise only, or counterclockwise only), and not in a back-and-forth motion. For example, the control unit may drive nail-contacting surface 26 in the single direction for a given period of time, for example, for more than 2 seconds, and/or less than 10 seconds (e.g., 2-10 seconds). Subsequently, the control unit drives the nail-contacting surface to move in a back-and-forth motion, as described hereinabove. For some applications, in response to the generation of the hole by nail-penetration device 20 being terminated (e.g., in response to a signal that is detected by a sensor unit, as described hereinbelow), the control unit resets the nail penetration device, such that the next time the nail penetration device is activated, the control unit drives nail-contacting surface 26 in the single direction for the given period of time.

For some applications, the control unit drives the nail-contacting surface to move in a rotating back-and-forth motion, such that there is a net rotation of the nail contacting surface in a given direction. For example, the control unit may drive the nail-contacting surface to, alternatingly, rotate through a first angle in a clockwise direction and through a second angle in a counterclockwise direction, the first angle being greater than the second angle, or vice versa. Thus, while driving the nail-contacting surface to undergo a back-and-forth motion, the control unit drives the nail-contacting surface to undergo a net rotation in the clockwise or counterclockwise direction. For some applications, by undergoing a net rotation, the nail-contacting surface facilitates the removal of nail debris from a hole that is created in the nail.

Alternatively or additionally to nail-contacting surface 26 moving in a back-and-forth motion, a sensor unit 27 facilitates detection of when the nail-contacting surface is in contact with, or in close proximity to, the nail bed, and the generation of the hole by nail-penetration device 20 is terminated in response thereto. For example, the sensor unit may include an ultrasound probe, and/or electrodes that are configured to detect impedance between surface 26 and a location on the subject's body, such as the patient's skin. Further alternatively or additionally, in response to sensor unit 27 detecting when the nail-contacting surface is in contact with, or in close proximity, to the nail bed, an indication is provided to the user that this is the case, for example, by illuminating an LED of the nail penetration device.

For some applications, sensor unit 27 includes a first electrode 29 disposed at the distal end of nail penetration device 20, e.g., disposed on nail-contacting surface 26. For some applications, the first electrode comprises an electrically-conducting coating of penetration element 24, e.g., a metallic coating, such as a gold or a copper coating. An electronic signal having given parameters is emitted via the first electrode. For example, the first electrode may emit a signal, the time-variation of the amplitude of the signal having a given shape. A second electrode 31 is placed on the subject's skin, in a vicinity of the nail. In response to the second electrode detecting the signal that is emitted via the first electrode, the generation of the hole by nail-penetration device 20 is terminated, since the detection of the signal indicates that nail-contacting surface has contacted, or is in close proximity to the nail bed.

For some applications, a pair of second electrodes 31 is placed on the subject's skin, in a vicinity of the nail (e.g., on the finger or toe, hand or foot, or arm or leg corresponding to the finger or toe being treated). The voltage between the pair of second electrodes is detected. When nail-contacting surface 26 has contacted, or is in close proximity to the nail bed, the current that is driven through first electrode 29 travels through the subject's skin, and affects the voltage that is detected between the pair of second electrodes. For example, the voltage between the pair of second electrodes may vary with time in accordance with the time variation of the current that is driven via the first electrode. In this respect, the pair of second electrodes is analogous to a pair of ECG electrodes. ECG electrodes are not placed on a subject's heart, but are used to detect the current that is conducted through the heart, by being placed on the subject's skin, and detecting changes in the voltage between regions of the subject's skin that are due to the current that is conducted through the heart. Similarly, although the second pair of electrodes is placed in the vicinity of the nail, but not on the nail bed, the voltage between the second pair of electrodes is used to detect a current that is driven into the nail bed. Typically, in response to the voltage between the second pair of electrodes varying in a manner that indicates that the nail-contacting surface 26 has contacted, or is in close proximity to the nail bed, the generation of the hole by nail-penetration device 20 is terminated.

For some applications, a signal is emitted via first electrode 29 that is configured to moderately stimulate a nerve ending (e.g., a pain receptor) of the subject, upon the first electrode coming into contact with, or being in close proximity to the nail bed. For example, a signal may be emitted via the first electrode, the signal having an amplitude of more than 3 mA and/or less than 20 mA, e.g., more than 7 mA and/or less than 15 mA, e.g., more than 8 mA and/or less than 12. Typically, the signal is an AC signal (e.g., having a frequency of more than 50 Hz and/or less than 1 kHz), such that capacitive current in effect passes into the tissue even when impedance between the first electrode that is still in the nail, and the nail bed, is too high to allow the driving current to be conducted into the nail bed. For some applications, the signal is a DC signal, and/or a combination of AC and DC.

If the subject is controlling device 20 (e.g., by pressing button 30, as described hereinabove), the generation of the hole by nail-penetration device 20 is terminated by the subject, upon the subject feeling a sensation (e.g., pain) that is indicative of the first electrode having come into contact with, or into close proximity to, the nail bed. If a user other than the subject (e.g., a healthcare professional) is controlling device 20 (e.g., by pressing button 30, as described hereinabove), the generation of the hole by nail-penetration device 20 is terminated by the user, upon the subject indicating to the user that the subject has felt a sensation (e.g., pain) that is indicative of the first electrode having come into contact with, or into close proximity to the subject's nail bed.

For some applications, when nail-contacting surface 26 comes into contact with, or into close proximity to, the subject's nail bed, stray electrons that are disposed on nail penetration element 24 are captured by the subject's skin (or stray electrons that are disposed on the subject's skin are captured by nail penetration element 24). The control unit of the nail penetration device is configured to detect the movement of the stray electrons, via second electrode 31, and to terminate the generation of the hole by nail-penetration device 20, in response thereto. For some applications, second electrode 31 includes a transistor, such as a metal-oxide-semiconductor field-effect transistor. The transistor is configured to amplify an electrical signal that is generated by the movement of the stray electrons. It is noted that for these applications, electrical current is not necessarily actively driven by the control unit of the nail penetration device through first electrode 29, disposed at the distal end of nail penetration device 20.

For some applications, the generation of the hole by nail-penetration device 20 is terminated in response to detecting a parameter that indicates that nail-contacting surface is in close proximity to (e.g., within 2-4 microns of) the nail bed, but not necessarily in contact with the nail bed. For example, one or more of the parameters described hereinabove may be detected, and a change in the detected parameter may be indicative of the nail-contacting surface being in close proximity to, but not necessarily in contact with, the nail bed.

For some applications, nail penetration device 20 moves penetration element 24 through fewer than 500 revolutions per minute, or fewer than 300 revolutions per minute (e.g., more than 50 and/or less than 200 revolutions per minute), or in cases in which the penetration element is moved in a back-and-forth motion, as described hereinabove, through fewer than 500 and/or more than 10 back-and-forth motion cycles per minute. For example, moving the penetration element 24 through fewer than 500 (e.g., fewer than 300, or fewer than 200) revolutions per minute, or through fewer than 500 cycles per minute, may reduce heating of the subject's nail relative to if the penetration element were moved at a faster rate. Alternatively or additionally, moving the penetration element 24 through fewer than 500 (e.g., fewer than 300, or fewer than 200) revolutions per minute, or through fewer than 500 cycles per minute, may reduce the release of nail debris into the region surrounding the nail, relative to if the penetration element were moved at a faster rate.

For some applications, the thickness of nail 28 is measured, and in response thereto, the depth to which to penetrate through the nail is determined. The depth to which the nail should be penetrated is inputted into the nail penetration device control unit. In response to the input, the control unit automatically terminates the generation of the hole by nail-penetration device 20 at the determined depth.

Reference is now made to FIG. 2, which is a schematic illustration of a substance dispenser 60 for administering a substance to the subject, as described in US Patent Application Publication 2010/0145373 to Alon, which is incorporated herein by reference, in accordance with some applications of the present invention. Typically, the dispenser dispenses a substance 62 for treating onychomycosis, for example, itraconazole (marketed as Sporanox®), and/or terbinafine (marketed as Lamisil®). For some applications, substance 62 includes antifungal agents and antibacterial agents for treating onychomycosis. Substance dispenser 60 includes a nozzle 64 for placing inside holes 52 on the subject's nail and administering the substance to the underlying nail bed. For some applications, holes of a specific size are generated by nail penetration device 20, and the nozzle is sized to match the size of the holes.

For some applications, holes 52 and nozzle 64 are sized such that when the nozzle is placed inside one of the holes, the nozzle forms a pressure seal with the nail. Subsequently, when the substance is dispensed from the dispenser, the substance is forced underneath the subject's nail. For some applications, the diameter of each hole 52 is 1-2 mm.

For some applications, substance 62 is administered to the subject via the holes using a different technique. For example, penetration element 24 may administer substance 62, through a lumen in the penetration element, via the holes, or a syringe may be used to inject the substance under pressure into a hole. Alternatively or additionally, the substance is sprayed onto the nail, such that the substance enters the holes.

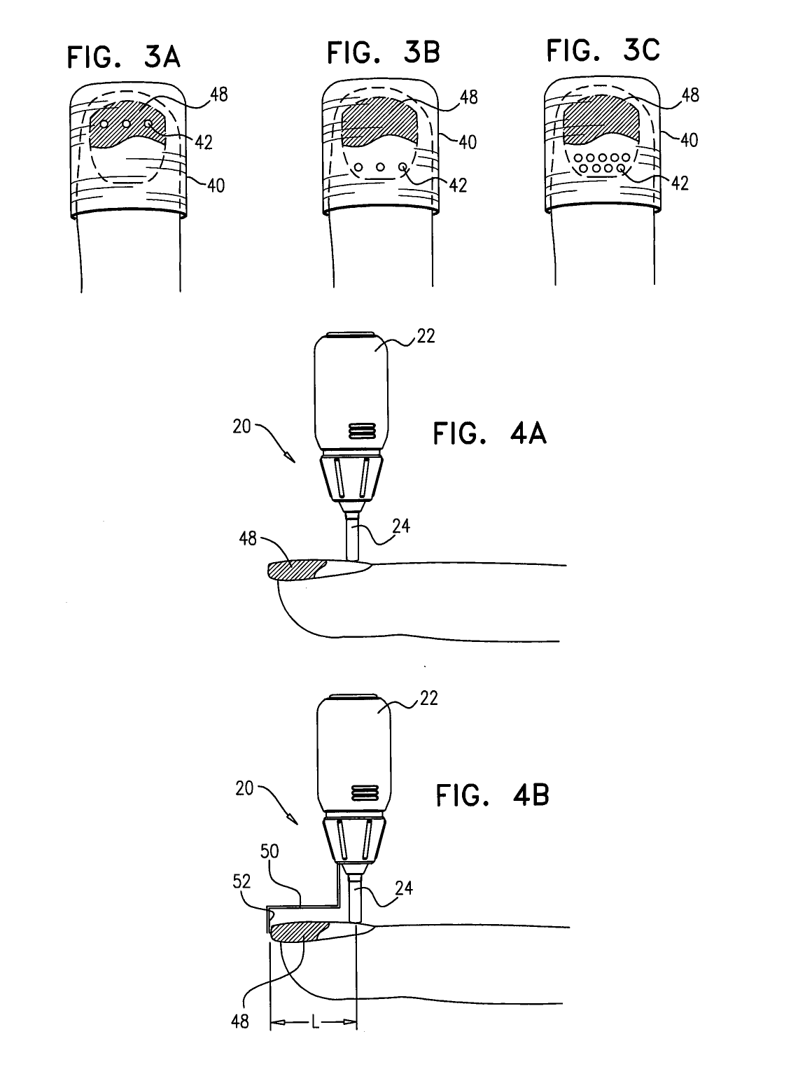

Reference is now made to FIGS. 3A-C, which are schematic illustrations of a template 40 for use with nail penetration device 20, in accordance with some applications of the present invention. Techniques shown in FIGS. 3A-C and described with reference thereto may be practiced in combination with the apparatus shown in FIGS. 1 and 2. For some applications, a plurality of holes are generated in the nail. For example, one to twelve holes may be generated in the nail. For some applications, in order to facilitate generation of the holes in suitable locations, a template is placed over the nail. The template defines a plurality of holes 42. The nail penetration device generates holes in the nail by penetration element 24 of the device being placed through respective holes in the template.

For some applications, a hole is generated at a distance from the distal end of the nail bed (i.e., the end that is closest to the tip of the finger or toe) that is less than 50% of the total length of the nail bed (e.g., within 5 mm of the distal end of the nail bed). For such applications, a template as shown in FIG. 3A is typically used, the template defining holes that are toward a distal end of the template (e.g., within 7 mm of the distal end of the template). For some applications, holes are generated toward the distal end of the nail bed, because this is where the nail is most affected by the onychomycosis.

Alternatively, holes are generated toward the proximal end of the nail bed, e.g., at a position that is proximal to where fungal growth 48 occurs, or at a proximal end thereof (e.g., more than 5 mm from the distal end of the nail bed). The holes are generated and a substance is administered to the nail bed via the holes, typically in accordance with a schedule (e.g., on a daily basis, or on a weekly basis). As the nail grows, the holes progress distally along the nail bed, and the substance is administered (e.g., by the subject) to locations along the nail bed that progress distally, thereby imparting an advancing line of healing to the nail bed. For such applications, a template as shown in FIGS. 3B-C is used, the template defining holes that are toward a proximal end of the template (e.g., more than 7 mm from the distal end of the template). The holes of the rows are disposed with respect to each other such that the center of at least one of the holes of a first one of the rows is not aligned with the center of any of the holes of a second one of the rows, when viewed along the direction of growth of the nail (i.e., the direction that the template provides for insertion of the finger or toe into the template, and/or the direction that is perpendicular to the opening of the template through which the finger or toe is inserted). Typically, the centers of the holes of the rows are disposed with respect to each other, such that there are holes in the nail corresponding to any horizontal location of the nail, across more than 50% (e.g., up to 100%) of the width of the nail. Further typically, the holes are disposed such that holes of one of the rows overlap with holes of at least one other row, across the width of the nail. Thus, when a substance is administered to the subject, via the holes, in accordance with a treatment schedule as described hereinabove, an advancing line of healing that covers over 50% (e.g., up to 100%) of the width of the nail is imparted to the nail bed, as the nail grows.

Reference is now made to FIG. 4A, which is a schematic illustration of hole 42 being generated in the subject's nail 28 at a position that is proximal to fungal growth 48 that is on the nail. As shown in FIG. 4A, for some applications, holes are generated proximal to the fungal growth, without nail penetration device 20 being guided by a template (as shown in FIG. 3B). Similarly, for some applications, holes are generated toward the distal end of the nail (as described with reference to FIG. 3A), without the nail penetration device being guided by a template.

Reference is now made to FIG. 4B, which is a schematic illustration of a placement element 50 for use with nail penetration device 20 (FIG. 1) and substance dispenser 60 (FIG. 2), in accordance with some applications of the present invention. For some applications, in order to guide the nail penetration device during the generation of holes in the nail, placement element 50 is used. For some applications, the placement element defines a surface 52 that is placed against the distal tip of the subject's nail or of the subject's finger or toe. The placement element is coupled to the nail penetration device, such that when surface 52 is placed against the tip of the nail or finger or toe, penetration element 24 is positioned at a suitable position for generating a hole in the nail. For some applications, a length L from surface 52 to the penetration element is 1-7 mm (e.g., for applications in which holes are generated toward the distal end of the nail). Alternatively, a length L from surface 52 to the penetration element is 7-20 mm (e.g., for applications in which holes are generated toward the proximal end of the nail). For some applications, length L is adjustable, such that the subject can adjust length L, as is appropriate for the subject's nails.

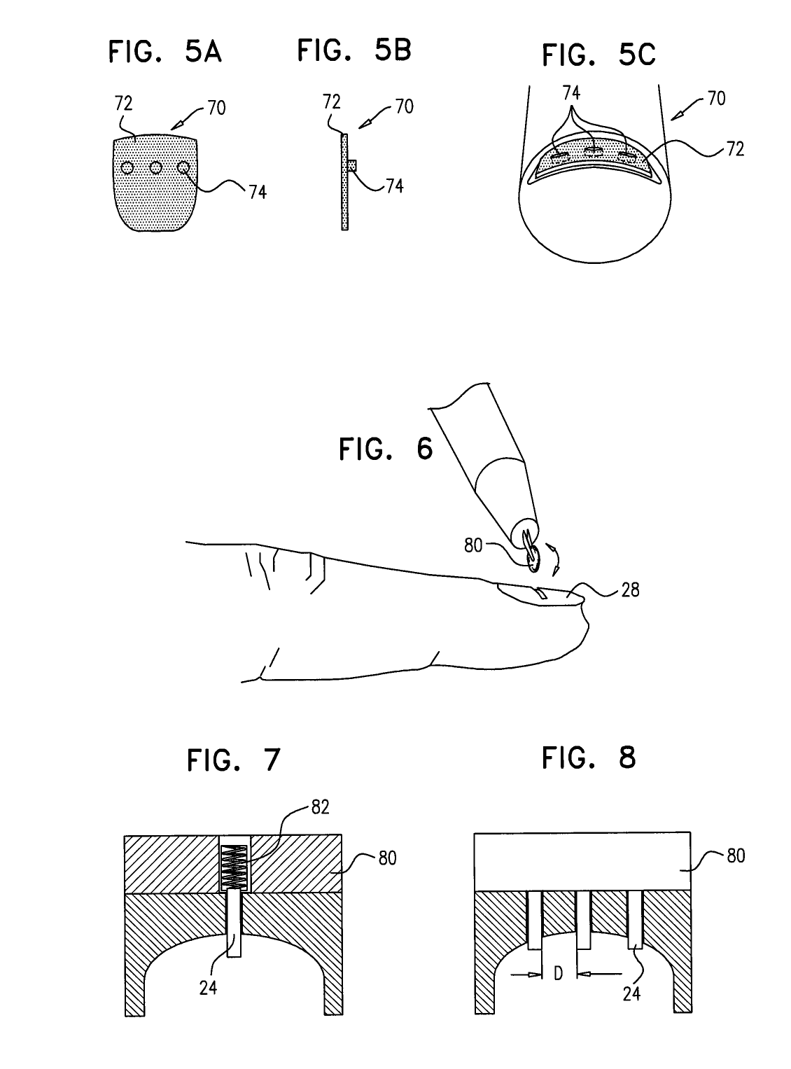

Reference is now made to FIGS. 5A-C, which are schematic illustrations of respective views of a plug unit 70 for placing over a hole in a nail, in accordance with some applications of the present invention. The plug unit typically includes a plurality of plugs 74 that protrude from a support 72. For some applications, the diameter of each of the plugs is 1-2 mm. Typically, the sizes of the holes that are generated in the nail and the sizes of the plugs are such that the plugs are held in place inside the holes, and seal the holes. For some applications a substance, e.g. substance 62 described hereinabove, is placed on a skin-contact surface of the plug that comes into contact with the nail bed. Thus, the substance is administered to the nail bed, via the plug.

FIG. 5C shows plug unit 70 when the unit has been placed on the subject's nail. For some applications, support 72 is a prosthetic nail. For some applications, the prosthetic nail is used in order to cover a nail that is suffering from onychomycosis. For some applications, the techniques described herein, for generating holes in a nail, and placing plugs 74 of plug unit 70 into the holes, are used for esthetic purposes, namely, in order to secure a prosthetic nail to the subject's nail, even though the subject is not suffering from onychomycosis or any other disease of the nail.

Reference is now made to FIG. 6, which is a schematic illustration of a rotating element 80 (e.g., a saw) for penetrating a nail, in accordance with some applications of the present invention. For some applications, the saw is a circular saw (as shown) having a diameter of more than 3 mm and/or less than 10 mm, e.g., about 5 mm. Techniques described with reference to FIG. 6 are generally similar to those described with reference to the other figures, except for the differences as noted. A nail-contacting surface of the rotating element typically rotates, while the nail-contacting surface is in contact with the subject's nail. The rotation of the surface alternates between being clockwise and counterclockwise. Typically, the surface alternates between rotating in a given direction (e.g., clockwise or counter-clockwise) and rotating in the opposite direction at a frequency of at least 2, 10, or 50 times per second. For some applications, the surface is rotated through clockwise-counterclockwise motion cycles at a frequency of less than 500 cycles per minute, e.g., in order to reduce heating of the nail, relative to if the direction in which the surface rotates were to alternate at a higher frequency.

Typically, the clockwise-counterclockwise nature of the motion of the nail-contacting surface prevents damage to the nail bed, which may otherwise be caused by the nail-contacting surface. When the nail-contacting surface makes contact with the flexible tissue of the nail bed, the nail-contacting surface simply moves the tissue of the nail bed, but does not cut through the tissue. However, when the nail-contacting surface is in contact with the nail, which is generally rigid, it cuts a hole or a groove through the nail. It is noted that although the rotating element shown in FIG. 6 is disc shaped, for some applications, the element is a different shape, such as rectangular or elliptical. Similarly, for some applications, the cutting element translates back and forth, instead of, or in addition to, rotating back and forth. As described hereinabove, with respect to nail penetration element 24 of FIG. 1, for some applications, the rotating element rotates in a back-and-forth motion, but undergoes a net rotation in one direction. Also, as described hereinabove, for some applications, the rotating element initially rotates in a single direction (e.g., for a given period of time, or for a given number of cycles), and subsequently, undergoes back-and-forth rotation.

As described hereinabove, for some applications, nail penetration element 24 moves in a back-and-forth motion, e.g., as described in US Patent Application Publication 2010/0145373 to Alon, which is incorporated herein by reference. For example, the nail-penetration element may be a flat disk, and/or a cylindrical element. For some applications, nail penetration element 24 and/or nail penetration element 80 is at least partially covered with an opaque cap (not shown) (a) so as to obscure the view that the subject has of the nail being penetrated by the penetration element, and/or (b) so as to shield the surroundings of the nail penetration device from nail debris.

FIG. 7 is a schematic illustration of a housing 80 for use with penetration element 24, in accordance with some applications of the present invention. Techniques shown in FIG. 7 and described with reference thereto may be practiced in combination with the apparatus shown in FIGS. 1 and 2. For some applications, in order to penetrate the nail of a finger or a toe, housing 80 is placed at least partially around the finger or the toe. For some applications, a mechanism 82 (e.g., a spring mechanism, as shown) couples penetration element 24 to housing 80. The mechanism controls the advancement of the penetration element, such that when the housing is pressed with a pressure that is below a threshold pressure, the penetration element is not activated to penetrate through the nail. Furthermore, the mechanism controls the advancement of the penetration element, such that when the housing is pressed with a pressure that is greater than the threshold pressure, the penetration element is activated to advance into the nail, and is applied to the nail with a constant pressure, irrespective of the extent to which the pressure with which the housing is pressed is greater than the threshold pressure.

Reference is now made to FIG. 8, which is a schematic illustration of housing 80, the housing being coupled to a plurality of penetration elements 24 for generating a plurality of holes in a subject's nail, in accordance with some applications of the present invention. Techniques shown in FIG. 8 and described with reference thereto may be practiced in combination with the apparatus shown in FIGS. 1 and 2. For some applications, two or more penetration elements are disposed in the housing, for example, such that a plurality of holes may be generated in the nail, e.g., simultaneously. For some applications, each of the penetration elements is spaced from an adjacent penetration element by a distance D that is greater than 1 mm, and/or less than 5 mm, e.g., 1 mm to 5 mm. Although, the penetration elements shown in FIG. 8 are disposed in a linear configuration with respect to one another, in accordance with some applications, a plurality of penetration elements are disposed in alternative configurations, such as in a triangular configuration. For some applications, in response to a sensor associated with a given penetration element detecting that the distal end of the penetration element is at the nail bed or in close proximity thereto, the generation of a hole by the penetration element is terminated (in accordance with the techniques described hereinabove). Alternatively, in response to a sensor associated with any one of the penetration elements detecting that the distal end of the penetration element is at the nail bed or in close proximity thereto, the generation of holes by all of the penetration elements is terminated.

It is noted that although the figures of the present application generally show the use of the apparatus and methods described herein on a fingernail, the scope of the present invention includes applying the apparatus and methods described herein to a toenail of the subject. Thus, the figures should not be interpreted as restricting the scope of the invention to being used with fingernails.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for cutting a hole in a nail of a subject, comprising:
an element having a nail-contacting cutting surface for contacting the subject's nail; and
a control unit configured to generate a hole in the subject's nail by moving the nail-contacting surface in a back-and-forth motion, while the nail-contacting surface is in contact with the subject's nail.

2. The apparatus according to claim 1, wherein the control unit is configured to move the nail-contacting surface in the back-and-forth motion, while causing the nail-contacting surface to undergo a net rotation in a direction selected from the group consisting of: clockwise and counterclockwise.

3. The apparatus according to claim 1, wherein the control unit is configured to move the nail-contacting surface in the back-and-forth motion, by moving the nail-contacting surface through back-and-forth motion cycles at a frequency of less than 500 times a minute.

4. The apparatus according to claim 1, wherein:
when the nail-contacting surface is initially activated, the control unit is configured to move the nail contacting surface in a single direction for at least two seconds, the single direction being selected from the group consisting of: clockwise and counterclockwise; and
subsequently, the control unit is configured to move the nail-contacting surface in the back-and-forth motion.

5. The apparatus according to claim 1, wherein the apparatus comprises a disc-shaped nail-penetration element, and wherein the nail-contacting surface comprises an outer edge of the disc-shaped nail-penetration element.

6. A method, comprising:
identifying a thickness of a nail of a subject;
determining a depth to which to penetrate through the nail, in response to the identified thickness;
inputting the determined depth into a control unit of a nail penetration device; and
generating a hole in the nail with the nail penetration device, the control unit being configured to automatically terminate the generation of the hole by the nail-penetration device at the determined depth.

7. Apparatus comprising:
a housing;
a plurality of nail penetration elements configured to generate respective holes through a nail of a subject, each of the penetration elements being coupled to the housing at a distance from an adjacent one of the penetration elements that is 1-5 mm;
a sensor unit, coupled to each penetration element and configured to sense a parameter at the distal end of the penetration elements; and
a control unit configured to terminate the generation of a hole by a given one of the penetration elements in response to the parameter that is sensed by the sensor unit indicating that the distal end of the given penetration element is in a vicinity of a nail bed underneath the nail.

8. The apparatus according to claim 7, wherein in response to the parameter that is sensed by any one of the sensor units indicating that the distal end of a corresponding penetration element is in the vicinity of the nail bed, the control unit is configured to terminate the generation of holes by of all of the penetration elements.

9. Apparatus for generating a hole in a nail of a subject, comprising:
a nail penetration device having a nail-contacting surface for contacting the subject's nail, and comprising a first electrode disposed on the nail-contacting surface;
a skin-contact sensor for detecting contact between the nail-contacting surface and a nail bed of the subject, the sensor comprising a pair of second electrodes disposable on skin of the subject in a vicinity of the nail; and
a device control unit configured to:
generate a hole in the subject's nail by moving the nail-contacting surface, while the nail-contacting surface is in contact with the subject's nail,
drive a current via the first electrode, the current having a given set of parameters,
detect a voltage between the second electrodes, and
stop the generation of the hole, in response to detecting a change in the voltage between the second electrodes that is indicative of the current that is driven via the first electrode having travelled through the subject's skin to the pair of second electrodes.

10. Apparatus for generating a hole in a nail of a subject, comprising:
a nail penetration device having a nail-contacting surface for contacting the subject's nail;
a skin-contact sensor for detecting contact between the nail-contacting surface and a nail bed of the subject, the sensor comprising an electrode disposable on skin of the subject in a vicinity of the nail; and
a device control unit configured to:
generate a hole in the subject's nail by moving the nail-contacting surface, while the nail-contacting surface is in contact with the subject's nail, and
stop the generation of the hole, in response to detecting a current via the electrode, the current being indicative of stray electrons conducted between skin of the subject's nail bed and the nail-contacting surface.

11. Apparatus for generating a hole in a nail of a subject, comprising:
a nail penetration device having a nail-contacting surface for contacting the subject's nail;
a skin-contact sensor for facilitating detection of contact between the nail-contacting surface and a nail bed of the subject, the sensor comprising an electrode disposed on the nail-contacting surface; and
a device control unit configured to:
generate a hole in the subject's nail by moving the nail-contacting surface, while the nail-contacting surface is in contact with the subject's nail, and
drive a current via the electrode, the current being configured such that, upon the electrode contacting skin of the subject's nail bed, the current stimulates nerve endings of the nail bed at a level sufficient to cause a sensation by the subject.

12. The apparatus according to claim 11, wherein the device control unit is configured to drive the current via the electrode by driving a current having an amplitude of more than 3 mA.

13. The apparatus according to claim 12, wherein the device control unit is configured to drive the current via the electrode by driving a current having an amplitude of more than 7 mA.

14. The apparatus according to claim 13, wherein the device control unit is configured to drive the current via the electrode by driving a current having an amplitude of more than 8 mA.

15. The apparatus according to claim 11, wherein the device control unit is configured to drive the current via the electrode by driving a current having an amplitude of less than 20 mA.

16. The apparatus according to claim 15, wherein the device control unit is configured to drive the current via the electrode by driving a current having an amplitude of less than 15 mA.

17. The apparatus according to claim 16, wherein the device control unit is configured to drive the current via the electrode by driving a current having an amplitude of less than 12 mA.

18. The apparatus according to claim 11, wherein the device control unit is configured to drive the current via the electrode by driving an AC current having a frequency of more than 50 Hz.

19. The apparatus according to claim 18, wherein the device control unit is configured to drive the current via the electrode by driving an AC current having a frequency of less than 1 kHz.

20.-37. (canceled)
